(19) United States
(12) Patent Application Publication
(10) Pub. No.: US 2003/0120142 A1
(43) Pub. Date: Jun. 26, 2003

(54) Method for identification and visualization of atrial tissue and therapeutical use thereof

(76) Inventors: Marc Dubuc, Longueuil (CA); Peter Guerra, St-Leonard (CA); Jean-Claude Tardif, Laval (CA)

Correspondence Address: OGILVY RENAULT, 1981 MCGILL COLLEGE AVENUE, SUITE 1600, MONTREAL, QC, H3A2Y3, CA

(21) Appl. No.: 10/253,927

(22) Filed: Sep. 25, 2002

Related U.S. Application Data
(60) Provisional application No. 60/325,198, filed on Sep. 28, 2001.
(60) Provisional application No. 60/395,998, filed on Jul. 16, 2002.

(51) Int. Cl. A61B 5/05

(52) U.S. Cl. 600/407

(57) ABSTRACT

The present invention relates to a method for visual identification of atrial tissue comprising the steps of visualizing a site of atrial tissue formation using a device adapted for visualizing and obtaining an image; and analyzing the image to determine the presence, location and/or distribution of atrial tissue in the site. The present invention also relates to a method for the treatment of atrial fibrillation in a patient, comprising the steps of identifying atrial tissue in a site of atrial tissue formation by introducing a device adapted for visualization into the site; and substantially ablating atrial tissue previously identified. The present invention further relates to a method for determining the shape of an atrial tissue formation comprising the step of identifying atrial tissue site potential indicative of the shape of the atrial tissue.

### BACKGROUND OF THE INVENTION

(a) Field of the Invention

This invention relates to a method for the identification of atrial tissue and method of treatment of atrial fibrillation using same.

(b) Description of Prior Art

The treatment of atrial fibrillation (AF) has evolved substantially in recent years, with increasing emphasis being placed on catheter-based approaches to therapy. Haïssaguerre and colleagues demonstrated that AF is actually initiated by atrial ectopics originating in the pulmonary veins (Haissaguerre M, et al., N Engl J Med. 1998;339:659-66) and that ablation of these foci could result in a cure of AF. The pulmonary veins (PV) were found to have unique electrophysiological properties, and recording studies suggest that certain PVs have longer sleeves of myocardial tissue thought to be responsible for the generation of these ectopic foci (Chen S A, et al., Circulation. 1999;100:1879-86). Anatomic evidence of sleeves of atrial tissue extending several centimeters into the PVs was described as early as 1966 by Nathan (Nathan H, Eliakim M., Circulation. 1966;34:412-22). More recent studies by Saito et al confirmed the presence of myocardial sleeves in the PV, with the longest sleeves being visualized in the superior veins (Saito T, et al., J Cardiovasc Electrophysiol. 2000;11:888-94).

Ablation of ectopic foci originating in the PVs was initially hampered by the lack of an adequate endpoint for the procedure, resulting in recurrences of AF. For this reason, elimination of PV potentials and PV electrical isolation was shown to be a more satisfactory endpoint (Haissaguerre M, et al., Circulation. 2000;101:1409-1417). Further, attempts at eliminating these potentials demonstrated that the conducting tissue and its breakthrough points were often asymmetrically distributed along the vein ostium (Hocini M, et al., Pacing Clin Electrophysiol. 2000;23:1828-31; Haissaguerre M, et al., Circulation. 2000;102:2463-5).

The methods known in the art to determine the localization of the tissue to ablate are deficient in the sense that they actually just provide an indication of an electric signal which is often inaccurate when the tissue is asymmetric.

It would be highly desirable to be provided with a method for identifying the anatomic substrate for the initiation for atrial fibrillation by visualizing sleeves of myocardial tissue in the pulmonary veins which could potentially serve as targets for ablation

### SUMMARY OF THE INVENTION

In accordance with the present invention there is provided a method for visual identification of atrial tissue in pulmonary veins, the method comprising the steps of:

a) visualizing a pulmonary vein using a device adapted for visualizing and obtaining an image;

b) analyzing the image to determine presence, location and/or distribution of atrial tissue in the vein.

The method in accordance with the a preferred embodiment of the present invention, wherein the device is selected from the group consisting of ultrasound probe, imaging device, optical coherence tomography device and magnetic resonance imaging.

The method in accordance with a preferred embodiment of the present invention, wherein the device is an ultrasound probe.

In accordance with the present invention, there is further provided a method for treatment of atrial fibrillation in a patient, the method comprising the steps of:

a) identifying atrial tissue in pulmonary veins by introducing a device adapted for visualization into pulmonary veins;

b) substantially ablating atrial tissue identified at step a)

wherein ablating atrial tissue results in the treatment of atrial fibrillation.

For the purpose of the present invention the following term is defined below.

The term “imaging device” is intended to mean any imaging device known in the art as a camera, ultrasound probe, optical device, optical coherence tomography device and magnetic resonance imaging.

The term “site of atrial tissue formation” is intended to mean any site where atrial tissues are susceptible to be formed in a patient and includes without limitation the pulmonary vein and the coronary sinus.

### BRIEF DESCRIPTION OF THE DRAWINGS

FIG. 1A illustrates IVUS and intracardiac recordings from 2 different PVs, where the smooth-contoured right inferior PV has no evidence of localized thickening;

FIG. 1B illustrates IVUS and intracardiac recordings from 2 different PVs, where the left middle PV is shown to have a crescent-shaped area of thickening;

FIG. 1C illustrates IVUS and intracardiac recordings from 2 different PVs, where in the same right inferior PV as in FIG. 1A, the recordings from the PV show only far field atrial signals;

FIG. 1D illustrates IVUS and intracardiac recordings from 2 different PVs, illustrating particularly high amplitude and high frequency potentials recorded from the same left middle PV as in FIG. 1B, as well as an initiation of AF from this vein. Eso=esophageal lead, RIPV=right inferior PV, LMPV=left middle PV, RA=right atrium, CS=coronary sinus, (d)=distal, (p)=proximal and (m)=mid.;

FIG. 2A illustrates IVUS images recorded during pullback from a left superior PV where no thickening was seen distally;

FIGS. 2B and 2C illustrate IVUS images recorded during pullback from the same left superior PV as in FIG. 2A, an area of thickening is visualized near a branch; and

FIG. 2D illustrates the 2 branches fused at the vein ostrium where the area of thickening was followed to the level of the left atrium.

### DETAILED DESCRIPTION OF THE INVENTION

In accordance with the present invention, there is provided a method for the identification of atrial tissue. One preferred embodiment of the present invention is using intravascular or intracardiac ultrasound.

In another embodiment of the method for identification of the present invention, an imaging device is used to identify the atrial tissue and a spray of saline is used to push the blood away from the imaging device.

In another embodiment of the method for identification of the present invention, the optical coherence tomography is used to provide identification of the atrial tissue.

In a further embodiment of the method for identification of the present invention, magnetic resonance imaging (MRI) is used to provide identification of the atrial tissue.

In accordance with the present invention, there is also provided a method for the treatment of atrial fibrillation in a patient.

Methods

Patient Population

We report 12 consecutive patients (5 women, 7 men) with a mean age of 41±8.9 years undergoing an electrophysiologic study for ablation of atrial fibrillation (AF). These patients had frequent episodes of paroxysmal AF resistant to medical therapy. None had structural heart disease. All had a transeosphageal echocardiogram prior to the procedure to document the absence of left atrial thrombus. Informed consent for the ablation procedure was obtained in all cases.

Electrophysiologic Study

The electrophysiologic study was performed using a decapolar catheter along the crista terminalis and in the coronary sinus; and quadripolar catheters in the His position, and at the right ventricular apex. Two transeptal punctures were performed in standard fashion using a Brockenbrough needle to allow mapping of the left atrium and PVs. Selective pulmonary venography was performed using hand injection of contrast material.

After the anatomy of the PVs was defined, mapping of spontaneous atrial ectopic beats and initiation of AF was performed by placing catheters initially in each of the right and left superior pulmonary veins, with the inferior veins being cannulated subsequently. In cases where there was insufficient atrial ectopy at baseline to determine the PV of origin, protocols of isoproterenol infusion (1 to 5 ug/minute), adenosine infusion (6 to 18 mg), rapid atrial pacing, and induction of AF followed by cardioversion were utilized to elicit and map the atrial ectopics responsible for the initiation of AF. The veins and their ostia were thoroughly mapped for any high frequency potentials (PV potentials). In the current study, only veins shown to have atrial ectopic beats initiating AF or AT were targeted for ablation.

Intravascular Ultrasound (IVUS) of the Pulmonary Veins

A 3.5 French, 30 mHz IVUS catheter (Boston Scientific) mounted on a guide wire was advanced under fluoroscopic guidance into each of the attainable pulmonary veins. The ostial diameter was documented, and distal recordings were performed to determine the extent of PV branching and to try to identify atrial tissue within each vein. a running audio commentary was performed during the advancement and the pullback of the IVUS catheter. The IVUS examinations were recorded onto S-VHS videotape.

In cases where wall thickening was identified by IVUS within the PVs, the mapping catheter was positioned to determine whether these regions showed high frequency potentials. Similar recordings were performed in regions without any evident atrial tissue to demonstrate the absence of such PV potentials.

Within each PV, the total vessel and lumen areas, and the minimal and maximal vessel and lumen diameters and circumference were measured. In cases where the vessel wall was asymmetric and showed localized thickening, the following measurements were obtained: the maximal thickening of the vessel wall, the percentage of the vessel circumference displaying this finding, as well as the length of this arc of thickened vein wall. The wall area at the site of thickening was calculated by subtracting the lumen area from the total vessel area.

Results

Of the 12 patients, all but 2 were found to have atrial ectopic beats originating in the PVs during the electrophysiologic study. Of the 2 patients in whom no pulmonary vein ectopic beats were found, one did not have any ectopy during the procedure despite the maneuvers listed previously, and one had AF that was initiated by an ectopic atrial tachycardia originating in the right atrium. The latter was successfully ablated. The remaining 10 patients all had atrial ectopic beats, atrial tacchycardia, and/or AF originating from their PVs. Two patients had previously had ablation attempts for AF, both were found to have recurrences originating from the same, incompletely isolated PV as during their original intervention.

Identification of Localized Thickening within the Pulmonary Veins with IVUS

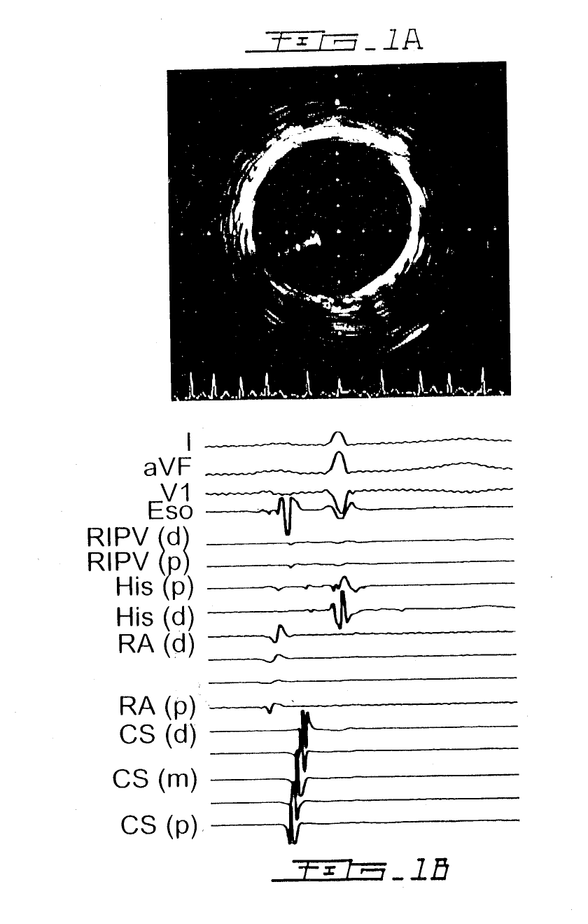

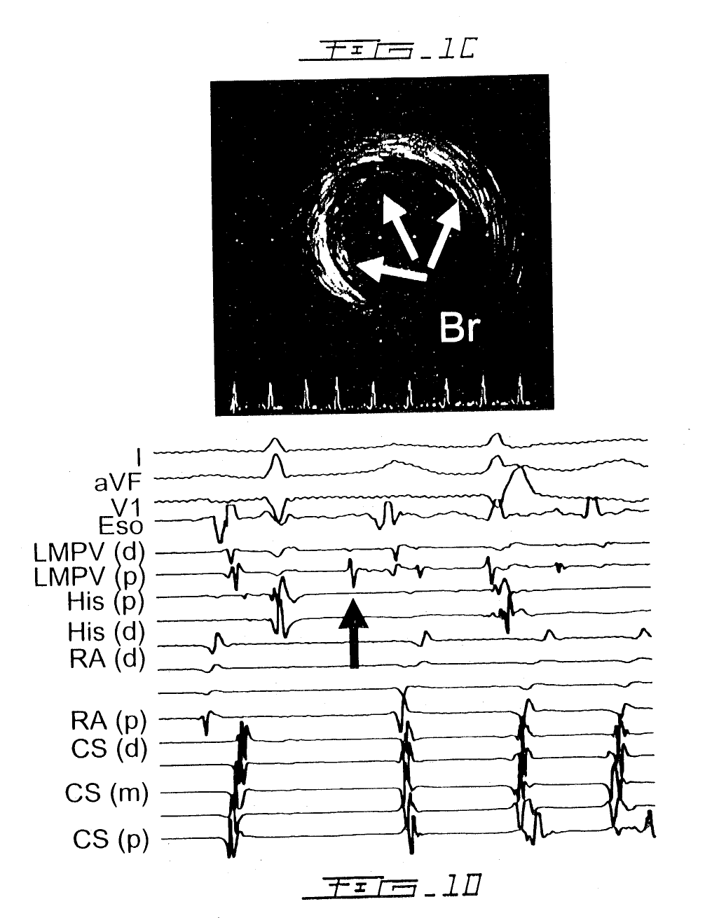

A total of 41 pulmonary veins were visualized using IVUS. Twenty-one of these veins had a smooth-contoured intima, with the vein thickness being very small and symmetric throughout (FIG. 1A). The vein wall thickness was less than 0.1 mm in these veins and their branches. This included 5 left superior PVs (LSPV), 5 right superior PVs (RSPV), 7 left inferior PVs (LIPV), 2 right inferor PVs (RIPV) and 2 left middle PVs (LMPV). However, the 20 remaining PVs (7 LSPV, 6 RSPV, 4 LIPV, 1 RMPV, 2 LMPV) were found to have a well-demarcated localized thickening of the vein walls which was moderately echogenic. This thickening was either almost circumferential, or more often asymmetric and seen as a crescent along a portion of the vein circunference (FIG. 1B).

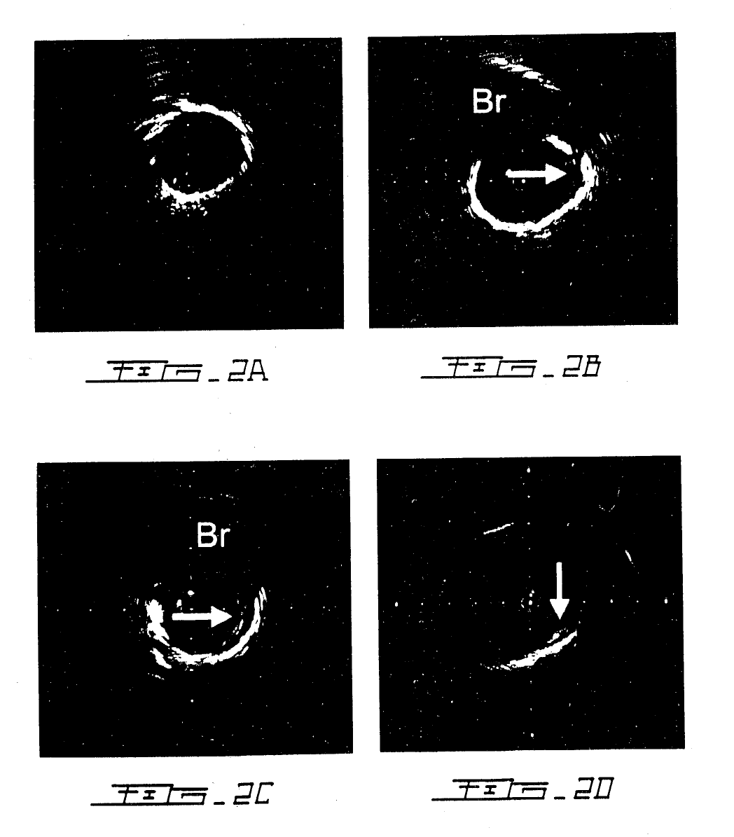

The width and length of these bands was quite variable, and their maximal thickness was 0.73±0.34 mm (range 0.30-1.31 mm, p<0.05; compared to the 21 smooth-contoured veins). This regional thickening comprised 38±20% of the veins' circumference (range 12-80%) for a mean of 13.3±10.5 mm arc of thickening (range 2.5-38 mm). The wall area at the site of maximal thickening ranged from 0.49 to 23.3 mm2 (mean 7.6 mm2, p<0.05 compared to the 21 smooth-contoured veins). These bands of tissue had a predilection for beginning near first or second order branches and through careful IVUS pullback could be traced to the PV ostium (FIG. 2). More distal examination in the PVs showed a disappearance of this tissue. Asymmetric regional contraction of the veins was seen predominantly in areas of marked thickening. These contractions were never present in the more distal PVs or in other proximal veins where no wall thickening was identified.

Total vessel area was 81.7±61.3 mm2 versus 88.5±53.7 mm2 for veins with and without focal thickening respectively. There was no significant difference between these veins' vessel diameters and circumference either.

Correlation between Appearance and Pulmonary Vein Potentials

Extensive mapping was performed in order to localize PV potentials and AF initiations in all veins during the study. Intracardiac recording in search of PV potentials was performed at three sites within the PVs that were cannulated: at the level of maximal vein wall thickening, proximal and distal to these areas of thickening, and at the ostium of each vein. Similar mapping was performed in veins without apparent thickening. IVUS allowed simultaneous visualization of both the mapping catheter and the PV tissue.

Of the 41 PVs studied, 21 failed to reveal any regional thickening, and none of these veins had any recordable PV potentials (FIG. 1C).

Twenty veins had regional thickening, and in these, the mapping catheter was placed directly on the thickened surfaces, In all cases, electrograms recorded at these sites showed the typical high frequency PV potentials initially described by Haïssaguerre (Haissaguerre M, et al., N Engl J Med. 1998;339:659-66) (FIG. 1D). More distal IVUS imaging showed an attenuation and then disappearance of this focal thickening, and electrograms recorded in these regions did not show any high frequency potentials, instead, only far field atrial signals were recorded.

In the present application, 10 veins were shown to be the site of origin of atrial ectopic beats and/or AF, and with one exception, all of these veins were found to have regional thickening as described (FIG. 1D). In the latter case, the patient had AF initiation from a right superior PV and this vein could not be cannulated with the IVUS catheter, so no correlation could be obtained. The two patients in whom AF initiation from the PVs could not be documented (notably the patient with a right atrial trigger) did not have regional thickening in any of the PVs that were visualized.

Discussion

The present application demonstrated the feasibility of performing IVUS in the pulmonary veins and also of identifying local anatomic abnormalities within the vein walls. IVUS showed areas of focal thickening, usually in crescent form along a portion of certain vein walls. These thickened areas showed contractile properties not seen more distally or in smooth-walled veins. Intracavitary recordings from all of these sites revealed pulmonary vein potentials that were likewise not recorded more distally or in smooth-walled veins. These factors show that the localized thickening, in fact, represents sleeves of myocardial tissue extending into the pulmonary veins. The anatomic observations made with IVUS concord with previous pathologic studies indicating preferential localization of these sleeves of tissue to the superior pulmonary veins (Nathan H, Eliakim M., Circulation. 1966;34:412-22; Saito T, et al., J Cardiovasc Electrophysiol. 2000;11:888-94), as well as the electrophysiologic observations made by Haïssaguerre and colleagues (Haissaguerre M, et al., Circulation. 2000;101:1409-1417; Haissaguerre M, et al., Circulation. 2000; 102:2463-5).

Because the previously described ablation protocol called for identification and targeting of a vein shown to trigger AF, it was possible to document that these triggers originated in veins with thickened walls. Eleven additional veins were found to have similar thickening and high frequency PV potentials, and these veins likely also bear the potential to induce AF. As PV isolation becomes a more desirable endpoint, all PV potentials become a target for ablation. As IVUS allows visualization of the myocardial sleeve responsible for these PV potentials, it provides an anatomic landmark for the ablation procedure and serves in the treatment of atrial fibrillation.

### EXAMPLE 1

Anatomy of the Atrial Musculature in the Pulmonary Veins as Defined by Intravascular Ultrasound

Ablation of the sleeves of atrial tissue in the pulmonary veins (PVs) can result in electrical isolation of these and a cure of AF. It is sought to define the anatomy of this arrhythmogenic atrial tissue using intravascular ultrasound (IVUS).

IVUS (3.2 French, 30 MHz catheter) was performed in the PVs of 12 patients admitted for AF ablation. In 20 PVs, contractile areas of asymmetric thickening with typical PV potentials were identified, representing sleeves of atrial tissue. With pullback, the length of these sleeves was measured at 34±18 mm (range 7.9-80). Three distinct patterns of atrial muscle distribution were identified. In type 1 (15 PVs), the tissue occupied a wide portion of the vein circumference but tapered off distally (from 20±10 to 8±5 mm). In 5 of these, the proportion of the PV circumference occupied by atrial tissue increased, as the PV tapered more rapidly than did the atrial tissue. Type 2 (4 PVs) had a narrow band of tissue at the ostium which became larger distally (11±7 to 17±6 mm). Type 3 (1 PV) was a linear band with no taper (10 mm throughout). Two PVs were found to have 2 discrete bands of atrial tissue at the ostium.

This in vivo demonstration by IVUS of atrial muscular sleeves in the PVs illustrates their variable anatomy. PVs with a narrow neck of tissue at the ostium (type 2) may be easily isolated. Conversely, PVs with thicker or multiple ostial bands (type 1) can require more extensive ablation. Therefore, knowledge of this anatomy can identify better targets for PV isolation procedures.

### EXAMPLE 2

Pulmonary Vein Isolation Guided by Intravascular Ultrasound: Identifying Targets for Atrial Fibrillation Ablation

Pulmonary vein (PV) isolation for atrial fibrillation (AF) currently consists of ablating the atrial extensions into the PVs and disconnecting them from the LA as assessed by distal recordings with loop catheters. It is sought to identify this atrial tissue at the PV ostium using intravascular ultrasound (IVUS).

Seven consecutive patients undergoing AF ablation had IVUS performed in their PVs using a 3.2 French, 30 MHz catheter (Boston Scientifics). Thirteen PVs were selected for isolation (6 right upper, 5 left upper, 2 left lower) on the basis of documented ectopy or AF from said vein. With IVUS pullback, each PV ostium was clearly located. IVUS identified areas of asymmetric thickening which were correlated with local PV potentials confirming that these were sleeves of atrial tissue. The maximal thickening of the atrial tissue at these sites 0.78±0.21 mm. The total area of this localized thickening was 8.5±5.6 mm² and comprised a 17±8 mm arc at the vein ostium. IVUS allowed visualization of the ablation catheter such that it could be positioned ostially at the sites where atrial tissue was identified. Ablation was performed along 41±13% of the PV ostium (range 23-64%). PV isolation as confirmed by loop catheter was demonstrated in 12/13 veins.

IVUS can identify PV ostia and the sleeves of atrial tissue which are the targets for ablation. This allows ablating close to the ostium and potentially limiting the area of lesion, which may reduce the risk of PV stenosis. IVUS can thus be a useful adjunct in AF ablation procedures.

### EXAMPLE 3

Visualization of Musculature in the Coronary Sinus Using Intravascular Ultrasound

Anatomic muscle bundles identified in the coronary sinus (CS) in animal and necropsy studies may be responsible for preferential left to right atrial conduction. Ablation of these bundles are an important component of left atrial isolation for the treatment of atrial fibrillation. It is therefore sought to identify these muscular bundles in vivo using intravascular ultrasound (IVUS).

An IVUS (3.2 French, 30 MHz) catheter was inserted in the CS of 9 patients undergoing electrophysiologic studies. Manual pullback was performed and distal and proximal images were obtained. Well-demarcated, echogenic wall thickening in the CS corresponding to these muscular bands was found in all cases. These bands were identified at the level of the LA at a mean of 41±14 mm from the CS os. These bands were occasionally circumferential, but more often formed a crescent along a portion of the CS wall. This thickening comprised 44±19% of the CS circumference (range 19-100%) for a total arc of muscle of 27±10 mm. The maximal thickness of these bands was 0.75±0.37 mm. Stimulation at the distal site of these muscular bands demonstrated atrial capture in all patients.

IVUS can identify muscular bundles in the CS in vivo. These bundles extend as far as the LA and may be the anatomic correlate that explains rapid left to right atrial conduction via the CS.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for visual identification of atrial tissue, said method comprising the steps of:
a) visualizing a site of atrial tissue formation using a device adapted for visualizing and obtaining an image;
b) analyzing said image to determine presence, location and/or distribution of atrial tissue in said site.

2. The method of claim 1, wherein said device is selected from the group consisting of ultrasound probe, imaging device, optical coherence tomography device and magnetic resonance imaging.

3. The method of claim 1, wherein said device is an ultrasound probe.

4. The method of claim 1, wherein said site of atrial tissue formation is selected from the group consisting of pulmonary vein and coronary sinus.

5. A method for treatment of atrial fibrillation in a patient, said method comprising the steps of:
a) identifying atrial tissue in a site of atrial tissue formation by introducing a device adapted for visualization into said site;
b) substantially ablating atrial tissue identified at step a)
wherein ablating atrial tissue results in said treatment of atrial fibrillation.

6. The method of claim 5, wherein said device is selected from the group consisting of ultrasound probe, imaging device, optical coherence tomography device and magnetic resonance imaging.

7. The method of claim 5, wherein said device is an ultrasound probe.

8. The method of claim 5, wherein said site of atrial tissue formation is selected from the group consisting of pulmonary vein and coronary sinus.

9. A method for determining the shape of an atrial tissue formation comprising the step of identifying atrial tissue site potential indicative of the shape of said atrial tissue.
